(19) United States
(12) Plant Patent Application Publication
(10) Pub. No.: US 2020/0288615 P1
(43) Pub. Date: Sep. 10, 2020

(54) STRAWBERRY PLANT NAMED 'DRISSTRAWSEVENTYTHREE'

(50) ;

(71) Applicant: Driscoll's, Inc, Watsonville, CA (US)

(72) Inventors: Katalin Monika Pakozdi, Maidstone (GB); Katarzyna Blake, Watsonville, CA (US); Maria Cruz Ayuso Hernandez, Watsonville, CA (US)

(73) Assignee: Driscoll's, Inc, Watsonville, CA (US)

(21) Appl. No.: 16/501,198

(22) Filed: Mar. 5, 2019

(51) Int. Cl. A01H 6/74

(52) U.S. Cl. PLT/208

(57) ABSTRACT

A new and distinct variety of strawberry plant named ‘DrisStrawSeventyThree’, selected for its conical fruit shape, juicy fruit flesh with good post-harvest qualities, large fruit size, healthy and strong plants, and productivity, is disclosed.

Latin name: Botanical classification: Fragaria x ananassa.

Varietal denomination: The varietal denomination of the claimed variety of strawberry plant is ‘DrisStrawSeventyThree’.

### BACKGROUND OF THE INVENTION

Cultivated strawberry is a hybrid species of the genus Fragaria that is grown worldwide for its fruit. Modern strawberry was first bred in Brittany, France, in the 18^{th} century by crossing Fragaria virginiana with Fragaria chiloensis. Strawberry fruit is an aggregate accessory fruit, with the fleshy part of the fruit being derived from the receptacle that holds the ovaries.

Strawberry varieties vary widely in color, size, shape, flavor, season of ripening, degree of fertility, and susceptibility to disease. Certain varieties vary in foliage, and some vary in the relative development of their reproductive organs. Typically, strawberry flowers appear hermaphroditic in structure, but function as either male or female. Generally, commercial production of strawberry plants involves propagation from runners and distribution as either plugs or bare root plants. Cultivation is either perennial or annual plasticulture. During the off season, strawberries can also be produced in greenhouses.

Strawberry fruit is widely appreciated for its characteristic bright red color, aroma, juicy texture, and sweetness. Strawberry fruit is a popular fruit that is generally consumed either fresh or in prepared foods, such as preserves and baked goods.

Strawberry is an important and valuable fruit crop. Accordingly, there is a need for new varieties of strawberry plants. In particular, there is a need for improved varieties of strawberry plant that are stable, high yielding, and agronomically sound.

### SUMMARY OF THE INVENTION

In order to meet these needs, the present invention is directed to an improved variety of strawberry plant. In particular, the invention relates to a new and distinct variety of strawberry plant (Fragaria x ananassa), which has been denominated as ‘DrisStrawSeventyThree’.

Strawberry plant variety ‘DrisStrawSeventyThree’ was discovered in Huelva, Spain in 2014, and originated from a cross between the female parent ‘DrisStrawThirtyFour’ (U.S. Plant Pat. No. 24,395) and the male parent ‘VES 030-092’ (unpatented). ‘DrisStrawSeventyThree’ was first asexually propagated via stolons in Valladolid, Spain in 2014.

‘DrisStrawSeventyThree’ was subsequently asexually propagated via stolons and underwent further testing in Huelva, Spain for three years (from 2015 to 2018). The present variety has been found to be stable and reproduce true to type through successive asexual propagations via stolons.

‘DrisStrawSeventyThree’ exhibits the following distinguishing characteristics when grown under normal horticultural practices in Huelva, Spain:

1. Horizontal attitude of hairs on pedicels;

2. Absent or very narrow width of band without achenes on fruit

3. Achenes level with surface of fruit; and

4. Calyx attachment inserted in fruit.

‘DrisStrawSeventyThree’ was selected for its conical fruit shape, juicy fruit flesh with good post-harvest qualities, large fruit size, healthy and strong plants, and productivity.

### DESCRIPTION OF THE DRAWINGS

This new strawberry plant is illustrated by the accompanying photographs. The colors shown are as true as can be reasonably obtained by conventional photographic procedures. The photographs are of plants that are five months from planting.

FIG. 1 illustrates whole fruit of variety ‘DrisStrawSeventyThree’.

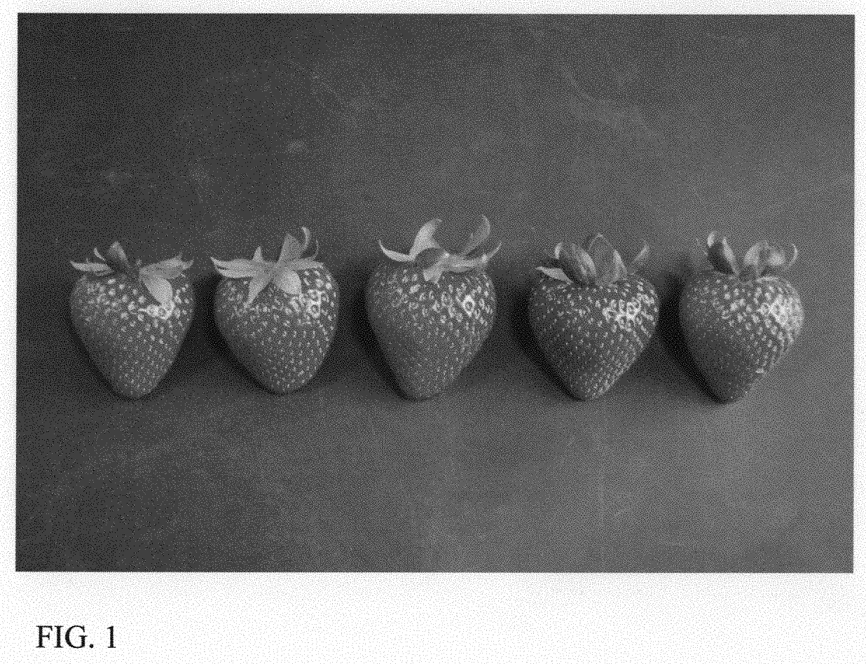

FIG. 2 illustrates the upper and lower surfaces of flowers of variety ‘DrisStrawSeventyThree’.

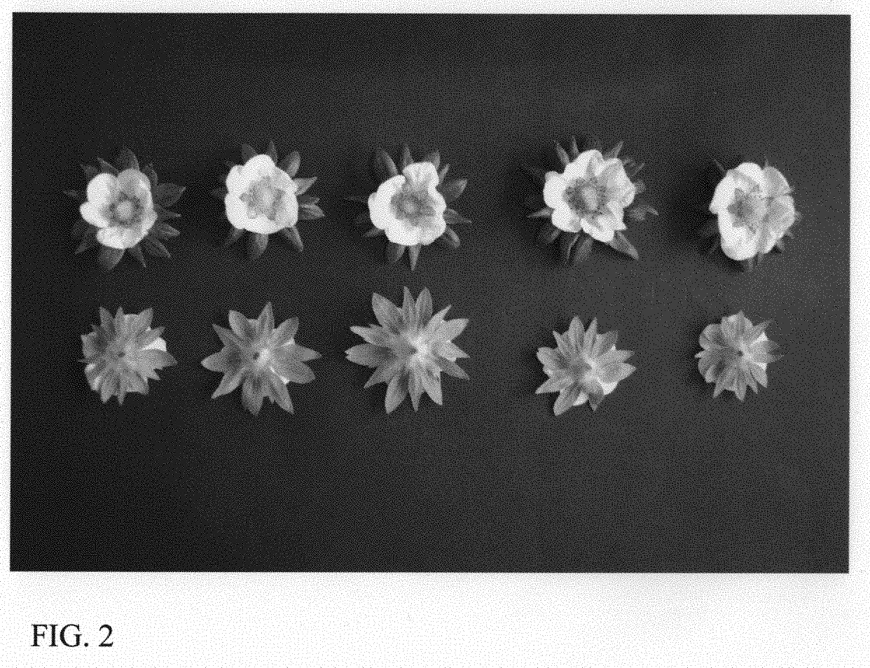

FIG. 3 illustrates the upper and lower surfaces of leaves of variety ‘DrisStrawSeventyThree’.

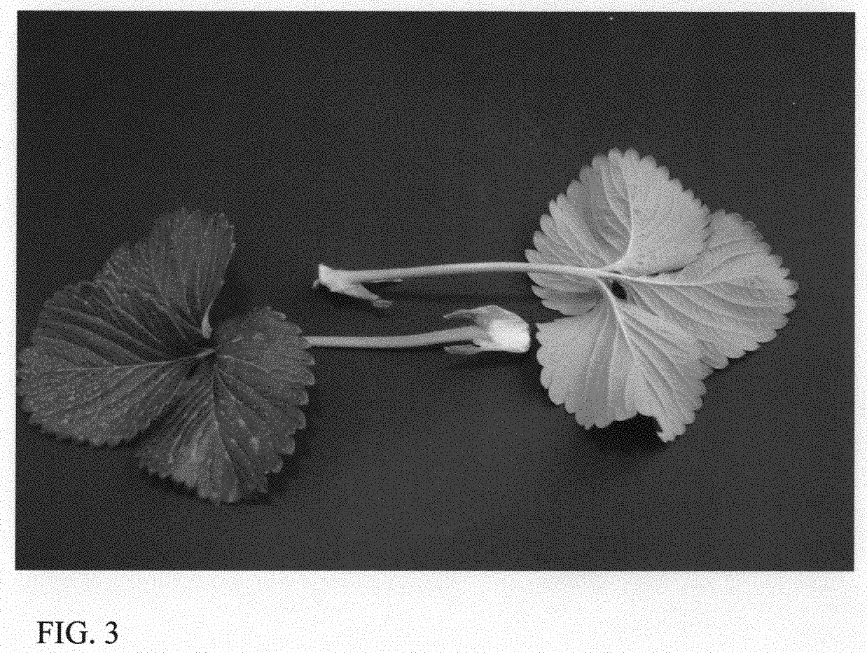

### DESCRIPTION OF THE NEW VARIETY

The following detailed descriptions set forth the distinctive characteristics of ‘DrisStrawSeventyThree’. The data which define these characteristics is based on observations taken in Huelva, Spain from 2015 to 2018. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawSeventyThree’ has not been observed under all possible environmental conditions. The botanical description of ‘DrisStrawSeventyThree’ was taken from plants that were five months from planting. The indicated values represent averages calculated from measurements of several plants. Color references are primarily to The R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
   Species.—Fragaria x ananassa.
   Common name.—Strawberry.
   Denomination.—‘DrisStrawSeventyThree’.
Parentage:
   Female parent.—Strawberry variety ‘DrisStrawThirtyFour’ (U.S. Plant Pat. No. 24,395).
   Male parent.—Strawberry variety ‘VES 030-092’ (unpatented).
Plant:
   Height.—26.7 cm.
   Diameter.—41 cm.
   Number of crowns per plant.—3.
   Growth habit.—Semi-upright.
Stolon:
   Average number of daughter plants per square foot.—4.
   Diameter at bract.—3.88 mm.
   Anthocyanin coloration.—Present.
   Stolon color with anthocyanin coloration present.—RHS 180B (Moderate red).
   Length.—40 cm.
Leaf:
   Number of leaflets.—Three only.
   Color of upper surface.—RHS 147A (Moderate olive green).
   Color of lower surface.—RHS N138C (Pale green).
   Venation pattern.—Cross-venulate.
   Leaf size (excluding petiole and stipules).—Medium.
   Variegation.—Absent.
   Terminal leaflets.—Length: 7.47 cm. Width: 7.68 cm. Length/width ratio: 0.97. Number of teeth/terminal leaflet: 19.1. Shape of base: Obtuse. Margin: Crenate. Shape in cross section: Concave.
   Petiole.—Length: 12.1 cm. Diameter: 2.70 mm.
   Attitude of hairs.—Horizontal.
   Bract frequency (number present on each petiole).—2.
   Color.—RHS 144A (Strong yellow-green).
   Petiolule.—Length: 8.7 mm. Diameter: 1.55 mm. Color: RHS 144C (Strong yellow-green).
   Stipule.—Length: 3.4 cm. Width: 9.9 mm. Anthocyanin color: RHS 144D (Light yellow-green).
Inflorescence:
   Position in relation to foliage.—Above.
   Peduncle.—Length: 22.2 cm. Diameter: 3.93 mm. Color: RHS 144C (Strong yellow-green).
   Pedicel.—Attitude of hairs: Horizontal.
   Flower bud.—Length: 17.98 mm. Diameter: 10.43 mm. Color: RHS 143B (Strong yellow-green).
   Flower.—Flower diameter (petal tip to petal tip on non-flattened flower): 29.9 mm. Typical and observed number of flowers per plant: 33. Arrangement of petals: Overlapping. Stamen: Present.
   Petal.—Length: 12.8 mm. Width: 12.9 mm. Length/width ratio: 0.99. Typical and observed petal number: 6.6. Color of upper side: RHS N155C (White). Color of under side: RHS N155B (White). Shape of apex: Rounded. Shape of base: Concave to convex. Margin: Entire.
   Calyx.—Diameter (sepal tip to sepal tip, measured on back of flower): 42.2 mm.
   Sepal.—Length (sepal tip to point of attachment to receptacle): 16 mm. Width: 6.5 mm. Typical and observed sepal number: 12.7. Color: RHS NN137B (Greyish olive green).
Fruit:
   Length.—45.2 mm.
   Width.—43.3 mm.
   Length/width ratio.—1.04.
   Fruit hollow length.—12.7 mm.
   Fruit hollow width.—4.3 mm.
   Fruit hollow length/width ratio.—2.93.
   Shape.—Conical.
   Color.—RHS N34A (Moderate red).
   Position of achenes.—Level with surface.
   Position of calyx attachment.—Inserted.
   Attitude of sepals.—Upwards.
   Color of flesh (excluding core).—RHS 32A (Vivid reddish orange).
   Color of core.—RHS 31C (Strong yellowish pink).
   Average weight per berry.—33 grams.
Production:
   Flowering interval.—Mid-November to mid-May.
   Harvest interval.—Late December/January to mid-May.
   Type of bearing.—Not remontant.
   Productivity.—0.850 kg to 1 kg of fruit per plant per season from 6.5-month-old plants when grown in Huelva, Spain.
Resistance to pests and diseases:
   Strawberry aphid (Chaetosiphon fragaefolii).—Moderately susceptible.
   Two-spotted spider mite (Tetranychus urticae).—Moderately susceptible.
   Powdery mildew (Podosphaera macularis).—Moderately resistant.

### COMPARISON WITH PARENTAL AND COMMERCIAL VARIETIES

‘DrisStrawSeventyThree’ differs from the female parent ‘DrisStrawThirtyFour’ (U.S. Plant Pat. No. 24,395) in that ‘DrisStrawSeventyThree’ has better fruit post-harvest quality and a later maturity as compared to ‘DrisStrawThirtyFour’.

‘DrisStrawSeventyThree’ differs from the male parent ‘VES 030-092’ (unpatented) in that ‘DrisStrawSeventyThree’ has better fruit post-harvest quality and an earlier maturity as compared to ‘VES 030-092’. Further, fruit of ‘DrisStrawSeventyThree’ are much larger in size than fruit of ‘VES 030-092’.

‘DrisStrawSeventyThree’ differs from the commercial variety ‘DrisStrawTwentySeven’ (U.S. Plant Pat. No. 23,400) in that fruit of ‘DrisStrawSeventyThree’ have absent or very narrow width of band without achenes, whereas fruit of ‘DrisStrawTwentySeven’ have very broad width of band without achenes. Further, fruit of ‘DrisStrawSeventyThree’ have achenes level with surface, whereas fruit of ‘DrisStrawTwentySeven’ have achenes below surface. Moreover, fruit of ‘DrisStrawSeventyThree’ have calyx attachment inserted in fruit, whereas fruit of ‘DrisStrawTwentySeven’ have calyx attachment raised from fruit. In addition, fruit of ‘DrisStrawSeventyThree’ have a strong adherence of calyx, whereas fruit of ‘DrisStrawTwentySeven’ have a weak adherence of calyx.

‘DrisStrawSeventyThree’ differs from the commercial variety ‘DrisStrawSixteen’ (U.S. Plant Pat. No. 22,247) in that petioles of ‘DrisStrawSeventyThree’ have a horizontal attitude of hairs, whereas petioles of ‘DrisStrawSixteen’ have an upwards attitude of hairs. Further, pedicels of ‘DrisStrawSeventyThree’ have a horizontal attitude of hairs, whereas pedicels of ‘DrisStrawSixteen’ have an upwards attitude of hairs. Moreover, fruit of ‘DrisStrawSeventyThree’ have calyx attachment inserted in fruit, whereas fruit of ‘DrisStrawSixteen’ have calyx attachment level with fruit. In addition, fruit of ‘DrisStrawSeventyThree’ have an upwards attitude of sepals, whereas fruit of ‘DrisStrawSixteen’ have an outwards attitude of sepals.

We claim:

1. A new and distinct variety of strawberry plant named ‘DrisStrawSeventyThree’ as shown and described herein.
